(19) United States
(12) Patent Application Publication
(10) Pub. No.: US 2004/0071780 A1
(43) Pub. Date: Apr. 15, 2004

(54) PACE-A microspheres for delivery of antigens

(76) Inventors: James W. Lillard, Stone Mountain, GA (US); Ravichandran Palaniappan, Atlanta, GA (US); Panduranga Rao Koritala, Naperville, IL (US)

Correspondence Address: Glenna Hendricks, Esq., Hendricks and Associates, P.O. Box 2509, Fairfax, VA, 22031-2509, US

(21) Appl. No.: 10/340,848

(22) Filed: Jan. 13, 2003

Related U.S. Application Data
(60) Provisional application No. 60/348,374, filed on Jan. 16, 2002.

(51) Int. Cl. A61K 39/00; A61K 38/22; B65B 1/00; A61K 9/14

(52) U.S. Cl. 424/489; 424/184.1; 514/2; 264/4

(57) ABSTRACT

Polycaprolactone- and chitosan-coated epichlorohydrin-crosslinked alginate (PACE-A) microspheres were prepared by a reproducible polymer dispersion technique that produced recombinant protein-containing particles averaging 8.2 μm in size. The PACE-A microspheres of the invention were coated with chitosan and polycaprolactone to increase the mechanical strength and stabilization and to modify the time of antigen release.

This application takes priority from Provisional Patent Application 60/348,374 filed Jan. 16, 2002.

### FIELD OF THE INVENTION

This invention relates to the use and novel preparation of polycaprolactone-coated and chitosan-coated epichlorohydrin-crosslinked alginate (herein referred to as “PACE-A”) microspheres as carrier systems for the mucosal or systemic delivery of both macromolecules and small molecules.

### BACKGROUND OF THE INVENTION

Nearly all widely used vaccines, with the exception of oral polio vaccine, are presently administered by systemic routes. In many cases these vaccines are effective in inducing systemic cell-mediated and antibody responses, but are poor at inducing mucosal immunity in humans who have not had a previous mucosal infection by the causative organism. Similarly, many therapeutic biologicals (i.e., peptides, enzymes, proteins) are delivered systemically to avoid degradation by mucosal secretions.

Delivery systems such as emulsions, liposomes and nanoparticles are widely regarded as carriers with adjuvant properties. Most of these delivery systems exhibit adjuvant properties due to their ability to release the antigen over a longer period than when the antigen is delivered in free form, while others are capable of modulating the immune system in addition to providing sustained or controlled-release properties. As with other peptide and macro-molecular therapeutics or biologicals, the rate of release of antigens from biodegradable microspheres was shown to be dependent mainly on degradation of the polymeric matrix.

A number of strategies are available to increase the efficacy of mucosally administered molecules. Common approaches involve the avoidance or modification of gastrointestinal secretions by the use of gastric inhibitors, protease and acid resistant films or encapsulation. An adjuvant activity has been demonstrated when muramyl dipeptide (MDP), liposomes or recombinant gram negative bacteria are administered orally. Immune stimulatory complexes (ISCOMS) confer immunogenicity on proteins delivered by the oral route. Small amounts of antigen in such structures can be rendered immunogenic. The method also provides some protection against enzymatic and acid degradation. The incorporation of antigens into liposomes or microparticles also provides some protection from harmful digestive secretions and thus allows the use of lower doses of antigen than when soluble antigen is administered.

The development of controlled release biological systems based on polymeric chemistries permits a sustained or pulsed release of their encapsulated contents. In recent years, biodegradable polymeric microspheres have received much attention for use to attain controlled release of vaccine antigens to eliminate the need for refrigeration and to reduce the number of immunizations and dosage requirements. The method targets antigens to microfold cells on mucosal surfaces or to antigen-presenting cells.

U.S. Pat. No. 4,744,933, which is incorporated herein by reference in its entirety, discloses a process of encapsulating bioactive materials.

Particulate delivery systems such as nanoparticles or microspheres also impart some adjuvant and protective properties to compositions when used as vehicles for oral administration of antigens. The adjuvant effect of microspheres made of poly (DL-lactide-co-glycolide) (DL-PLG) copolymer containing Staphylococcal enterotoxin B (SEB), when subcutaneously (sc) injected into mice, was comparable to effect of Freund's complete adjuvant (FCA). However, microspheres did not induce inflammation and granulomata in the mice. Ovalbumin (OVA), a poor immunogen when entrapped in DL-PLG microparticles, induced significantly higher levels of IgG antibodies in mice following primary immunization than did OVA in FCA.

U.S. Pat. No. 5,453,368, which is incorporated herein by reference in its entirety, discloses a method for encapsulating a biological substance using biocompatible microcapsules. Additionally, it discloses the coating of the microcapsules with solution of a soluble organic polymer in organic solvent. U.S. Pat. No. 5,879,713, which is incorporated herein by reference in its entirety, teaches the targeted delivery of small molecules such as nucleic acids and peptides.

Even though many of the methods addressed above have shown limited success, mucosal delivery systems and conversion of multiple into single dose(s) vaccine systems using the controlled delivery concept are still a challenge. This invention describes a novel preparation technique for the generation of PACE-A microspheres that provide for the controlled and protected release of its active agents.

### SUMMARY OF THE INVENTION

The present invention provides novel methods for the preparation and modification of biodegradable microspheres. These microspheres were characterized by size, surface charge and morphology. One of the difficulties in vaccine development is that only microparticles <10 μm can be taken up by microfold (M) cells. Polycaprolactone- and chitosan-coated epichlorohydrin-crosslinked alginate (PACE-A) microspheres were prepared by a reproducible polymer dispersion technique that produced recombinant protein-containing particles averaging 8.2 μm in size. Current alginate microspheres are only as small as 100 to 1000 microns in size. The present invention, in entrapment and loading studies with bovine serum albumin (BSA), showed that >80% entrapment efficiency and 18% of loading per weight of the microspheres could be achieved. The new methods of making formulations containing PACE-A microspheres also effectively provided for the programmed time-release of the entrapped protein antigen. The PACE-A microspheres of the invention were coated with chitosan and polycaprolactone to increase the mechanical strength and stabilization and to modify the time of antigen release. In a preferred embodiment, PACE-A microspheres coated with the polymers studied gave microspheres in the size range of 4 μm to 12 μm with the majority in the size range of 8 μm to 10 μm. PACE-A microspheres were coated 3 times with chitosan gave best in vitro release of the drug. Chitosan coating also yielded improved release.

### DETAILED DESCRIPTION OF THE INVENTION

Polymeric microspheres have been widely investigated as drug delivery systems because of their versatile route of administration (orally or parenterally). Using methods of the invention, the incorporated drug is protected from inactivation and provides controlled release of the drug. These microspheres are also used for systemic, subcutaneous, peritoneal, dermal and intramuscular administration.

The use of particulate carriers such as microspheres as oral delivery systems would be ideal for use in providing therapeutic agents at non-toxic levels. Early studies in mice have demonstrated the induction of enhanced systemic antibody responses following parenteral administration of antigens entrapped in polylactide glycolide microparticles. The potent immune response induced by these microspheres could be attributed to efficient antigen presentation due to phagocytosis by antigen presenting cells.

Since it has been shown that orally administered particles can be internalized by Peyer's patches (PP) of the gastro intestinal tract, other studies have investigated the potential of microparticles as oral vaccine delivery systems. Studies have demonstrated the induction of enhanced serum and secretory IgA antibody responses in rodents following oral administration of antigens entrapped in microparticles. Although the precise parameters controlling particle uptake into the PP are poorly understood, several studies have shown the importance of particle size on uptake. In particular, the optimal size for a particle to be internalized by M cells is 1-10 μm in diameter. In view of these findings, attempts were made in the present investigation to prepare alginate microspheres in the size range of 1 to 10 μm in diameter.

In order to develop controlled-release microspheres or vaccine, it is desirable that the delivery system degrade slowly (i.e., 8 to 70 days) to release the entrapped protein or nucleic acid (e.g., macromolecule) or polysaccharide (e.g., small molecule) for efficient presentation of immunogen to inductive sites of the immune system or of the therapeutic to target tissue of the host system. This, in the case of antigen presentation, insures a longer duration of protective antibody and more effective immunization. Even though a few protein delivery systems have been recently shown to deliver antigens in a controlled fashion, these systems are either costly or involve preparatory methods using organic solvents that can degrade the vaccine antigens or other biologically active agents.

Because presently available alginate microspheres are only effective for delivery of peptides and proteins over 8-10 days, there is a need to modify the surface and internal characteristics of microspheres for extended release. PACE-A microspheres disclosed herein were prepared by a novel polymer dispersion technique in which chitosan was used to prevent the interaction of a protein (BSA) with the internal matrix and polycaprolactone (PCL) was used to coat the microparticles for extended time release for a programmed release rate.

Other (prior art) alginate microspheres are bead or hydrogels that are not as efficient in targeting microfold cells of the Peyer's patch and other mucosal inductive sites (i.e., nasal tract, reproductive tract, lung, and lymph nodes. The prior art microspheres are cross-linked with calcium chloride. The PACE-A microspheres of the invention are cross-linked with epichlorohydrin, which yields a more stable physical structure and precise control for optimal size. Prior art microsphere technologies use poly-lysine for coating. In preparing the novel PACE-A microspheres, chitosan was used to separate macromolcules and smaller molecules carried by the particles from the alginate matrix. The resulting PACE-A microspheres yield unique microparticles for the delivery of biologicals and therapeutics across mucosal surfaces and the periphery.

Surface morphology and antigen release patterns were significantly influenced by the method of sphere formation and polymer coating. The chitosan-coated microspheres released the entrapped antigen within 8 days. Addition of PCL coating prolonged the release up to 70 days. Circular dichroism spectrum analysis confirmed that the molecular weight and conformation of the model protein antigen remained unaltered by the entrapment procedure. Taken together, these results show that this novel preparation method yields biodegradable microspheres of <10 μm that efficiently release their contents in a dose and time dependent fashion.

Materials and Methods:

Protein and Polymerization Reagents

For use in exemplifying the invention, BSA was chosen as a model protein because it is well-characterized, readily soluble in water, and large in size (molecular weight=68,000 Dalton). BSA (Sigma, St. Louis. USA), PCL and distilled methyl methacrylate (MMA) and Poly MMA (SRL, India) were used in the polymerization studies. Sodium alginate (Sigma), calcium chloride and epichlorohydrin (SD fine chemicals India) was used as a cross-linking agent. Chitosan (Sigma) and PCL (Sigma) were used to protect and encapsulate the PACE-A microspheres to limit degradation and for programmed time release of BSA.

Preparation of PACE-A Microspheres

The PACE-A microspheres were prepared by a novel polymer dispersion method. Poly MMA was prepared by polymerizing distilled MMA using redox-initiation technique with potassium persulfate (K₂S₂O₈) and sodium bisulfite (NaHS0₃) in an aqueous medium (H₂O). Alginate was dissolved in H₂O and dispersed in the poly MMA solution in organic medium using a homogenizer. The dispersed alginate was then made alkaline by adding 1N NaOH and crosslinked using a epichlorohydrin solution by adding 12% of epichlorohydrin in dispersed medium and allowing cross-link for 20 minutes. The polymer dispersion was homogenized thoroughly using Ultratorex homogenizer. The resulting PACE-A microspheres was then precipitated and retrieved by removing the poly MMA by washing 5 times with 1N toluene followed by washing 5 times with 1N acetone. Microspheres were further stabilized by curing with different concentrations of calcium chloride ranging from 1% to 15% (e.g. 1, 2, 4, 8 and 12%). The calcium chloride was added drop wise with continuous stirring. The microspheres were finally rinsed with H₂O, dried at room temperature and stored as a powder at 4° C.

PACE-A Protein Loading

Proteins (e.g., macromolecules) or polysaccharides (e.g., small molecules) can be incorporated onto microspheres by two methods, namely, swelling or in situ. Both methods were effective. In one case, BSA was added at 0° C. to the alginate solution prior to the addition of epichlorohydrin. In using the swelling method, a known amount of placebo microspheres were suspended in a known concentration of BSA. After 24 hours of incubation, the microspheres were removed by centrifugation, dried at open air and stored at 4° C. for further use.

Coating of Microspheres

PACE-A microspheres were coated with chitosan by placing the microspheres in chitosan coating solution (4% chitosan solution in acetic acid) with shaking in a conical flask for 15 minutes. The resulting chitosan-coated PACE-A microspheres were separated from the chitosan coating solution by draining the supernatant after centrifugation. The single-coated dried microspheres were then recoated two additional times with the chitosan coating solution.

For purposes of extending the time of release of BSA, the chitosan-coated microspheres were subsequently coated again with PCL. A known quantity of microspheres were allowed to swell in methanol at 4° C. After decanting the methanol, the microspheres were coated with various concentrations of PCL using 5%, 10% or 20% w/v of PCL in dichloroethane. Microspheres with additional coats of PCL were prepared in a similar manner. The resulting PCL- and chitosan-coated epichlorohydrin-crosslinked alginate (PACE-A) microspheres were strained, air dried and stored at 4° C.

Quantification and Characterization of Protein Loading

PACE-A microspheres were weighed and digested in the citrated tris buffer at 37° C. for 24 hour. Samples were filtered through a 0.45 μm Millipore filter and diluted with citrated tris buffer and assayed by Lowry's protein assay method using spectrophotometer (Shimadzu UV-2100S) for BSA content.

Circular dichroism spectrum analysis was used to assess the regularity of arrangements of the molecular assemblies. The conformations of the helical structures of the released BSA were examined by measuring the CD spectra with a Jasco J-500 spectrometer (Japan). Standard BSA and BSA released from microspheres were prepared in isotonic PBS (pH 7.4) and diluted appropriately. The spectrums were normalized for the solvent used.

Particle Size and Morphology Analysis

All particles were analyzed for their particle size by laser diffraction using Malvern particle size analyzer UK. The PACE-A microspheres were dispersed in HPLC grade water (SRL, Bombay, India) and analyzed for particle size. The surface morphology of the PACE-A microspheres were characterized by scanning electron microscope (SEM). The microspheres for SEM analysis were prepared by dispensing the dried microspheres onto one side of a double adhesive tape, which was stuck to an aluminum stub. The stubs were then coated with gold using Polaron SC S00-sputter coater to a thickness of 20-30 nm. The samples were then introduced into the specimen chamber of a Leica scanning electron microscope and examined for surface morphology.

The infrared spectra of the different stages of the PACE-A microsphere formulation was obtained by first mixing 1 mg of the finely powdered sample with 100 mg of dried potassium bromide powder. Next, the Infrared spectra of the samples were assayed using a Fourier transformed infra red spectrometer (Nicolet 20DXB Madison, Wis.). The surface charges of the microspheres were analyzed by measuring their zeta potential. The PACE-A microspheres were dispersed in 1 mM KCl pH 5.0 and analyzed by laser doppler anemometry, using a zeta meter (Malvern ZetaSizer 4, Malvern Instruments Ltd., Malvern, UK)

Thermal Analysis

Differential scanning calorimetry studies of alginate, alginate microspheres, PACE-A microspheres, BSA loaded microspheres, chitosan-coated PACE-A microspheres and chitosan- and PCL-coated PACE-A microspheres were carried out using Dupont-2000 differential scanning calorimeter. Dry samples weighing about 1 mg were placed in a pan and a lid was crimped to maximize the contact between the sample and the pan. The samples were heated from 30° C. to 250° C. at a constant heating rate of 10° C. min⁻¹ in the atmosphere of nitrogen. Thermal gravimetric profiles of the microspheres was carried out using the same instrument.

In Vitro Protein Release Studies of Uncoated, as Well as Chitosan- and PCL-Coated PACE-A Microspheres

In vitro release studies of PACE-A microspheres after incorporation of BSA and polymer coating were carried out in phosphate buffered saline pH 7.4 at 37° C. Microspheres (100 mg) were suspended in a 100 ml of phosphate buffer. For the uncoated PACE-A microspheres, aliquots were centrifuged and supernatant were removed and filtered through a 0.45 μm Millipore filter every hour. Next, the BSA content was determined by to Lowry's protein assay method. In the case of polymer-coated microspheres, samples were collected everyday. The PBS was replaced, after sampling, to mimic infinite sink conditions of the body.

Results: Effect of Dispersing Media Composition in the PACE-A Microspheres Characteristics

The type and concentration of dispersing agent played a significant role in the formation of the PACE-A microspheres. For example, the 10% poly MMA solution did not produce the desired particle size (1-10 μm); instead, this poly MMA solution produced particles ranging from 14 to 20 μm. However, a 12% poly MMA solution produced the ideal particle size range of 3 to 10 μm (average size 8 μm). Further increase in percentage of poly MMA does not have any additional advantage in reducing the particle size. Since the 12% poly MMA solution produced the desired PACE-A size, subsequent studies used this optimal concentration.

To study the effect of alginate concentration on the formation of microspheres, the 12% poly MMA and 12% epicholorohydrin solutions were mixed with various concentrations (0 to 100 mg) of sodium alginate. Microspheres in the size range of 1-12 μm were produced by using a 15% sodium alginate. At concentrations of less than 6% sodium alginate the yield of microspheres was found to be very low. At concentrations above 15% the sodium alginate solution was highly viscous and so could not easily be used for microsphere preparation. Hence, in the subsequent studies, up to 15% solution was prepared by placing the sodium alginate in water bath for 18 hours and homogenizing the viscous solution during the preparation of microspheres. The 14% sodium alginate solution provided the highest yield of microspheres.

The viscosity of the sodium alginate solution also had a significant influence on the morphology of the microspheres. PACE-A microspheres became smoother and more spherical with increasing concentrations of sodium alginate solution. However, At concentrations of 10% to 15% sodium alginate the ideal size of microspheres were produce, while the optimum yield was achieved with the 14%. In the subsequent studies, the 14% solution of sodium alginate was usually used for the preparation of PACE-A microspheres.

Effect of Stirring Speed on Particle Size of Microspheres

The stirring speed of homogenizer played a significant role in producing the optimal PACE-A microsphere size. Using the 12% Poly MMA, 14% sodium alginate, and 12% epichlorohydrin solution, microsphere formation was modulated by varying homogenizer stirring speed from 1000 to 10,000 rpm. At 1000 rpm aggregates were formed, which finally formed into a gel matrix.

Increasing the homogenizer speed to 5000 rpm yielded 20 to 35 μm microspheres. At between 5000 and 7000 rpm, 8 to 20 μm PACE-A microspheres formed. Further increasing the homogenizer stirring speed to 8000 to 10,000 rpm produced uniform microspheres ranging from 1 μm to 10 μm in size. Further increase in speed does not have any significant effect on size reduction. Further studies were carried out at 10,000 rpm.

Effect of Crosslinking Agent Concentration on the Formation of PACE-A Microspheres.

Epichlorohydrin was selected as the alginate polymer cross-linker because of its high efficiency and low toxicity compared to other cross-linking agents. Microspheres were not obtained when the percentage of epichlorohydrin was below 5%. Even though 6% to 7% epichlorohydrin produced microspheres, the yield was low compared to production when an 8% to 12% solution of epichlorohydrin was used. At the 15% concentration of epichlorohydrin smooth and free flowing PACE-A microspheres were produced. At more than 15% epichlorohydrin, the polymer dispersion (poly MMA and sodium alginate) solution forms a gel instead of microspheres. Hence, a second crosslinking agent was used.

CaCl₂ is a well-known gelling agent for alginates. Various concentrations of CaCl₂ ranging from 2% to 15% were added with stirring. Further increases in CaCl₂ concentration did not improve PACE-A microsphere stability and concentrations >20% inhibited microsphere formation.

PACE-A Microsphere Protein Entrapment and Loading Efficiencies

The amount of BSA successfully entrapped in PACE-A microspheres was determined by digesting microparticles in the citrated tris buffer. Increasing the amount of BSA during the preparation of PACE-A microspheres increased the protein loading of the microspheres from 4% to 18%. Increasing the concentration of BSA from 18%, did not significantly increase protein loading. A maximum of 18.46% BSA loading in the PACE-A microspheres could be achieved. Beyond 18% loading of protein, the spheres became aggregated and morphologically malformed. The protein loading data indicated that the percentage of entrapment by in situ methods was two fold greater than when the swelling method was used. Hence, for further investigations PACE-A microspheres were loaded by the in situ method.

PACE-A Microsphere Polymer Coating

In the studies, alginate microspheres were coated with the polycation, chitosan, to eliminate protein—alginate matrix interactions. The chitosan coating also improved the surface morphology, mechanical stability and the release pattern of the entrapped protein.

Poly Caprolactone Coating of PACE-A Microspheres

PCL has been widely investigated as a matrix material for the fabrication of slow release drug delivery systems. PCL's biocompatibility has also been well established. PCL has been used with many polymers and has been useful for manipulating the rate of release of nanoparticles. While alginate microspheres normally degrade in 8 to 15 days, coating with PCL extended the protein release period of the PACE-A microspheres.

There was no marked difference in size range between a single and double coating of PCL with alginate microspheres. Even though there was no difference in the particle size, there was a significant difference in the rate of release of BSA among these systems. Further, the coating thickness of PCL correlated to duration of in vitro release.

Morphologies of Uncoated, Chitosan and/or PCL Coated PACE-A Microspheres

It is evident from SEM that the microspheres modified by PCL and/or chitosan coating appeared spherical and uniform compared to the uncoated alginate microspheres. Dramatic changes of PACE-A microspheres surface characteristics occurred with time. The PACE-A microspheres were smooth and spherical in nature. The surface of the microspheres changed from smooth and spherical to rough, non-spherical and vacuolated after 9 days of protein release. PCL coating provided an additional barrier by blocking the pores of the chitosan coated microspheres, which slowed the release of the entrapped BSA.

Infrared Spectra of Uncoated and Coated Microspheres

The infrared spectrum of microspheres showed the cross-linking efficiency of polymers as well as the differences in chitosan- and PCL-coated microspheres. Infrared spectrum of sodium alginate shows an absorption band at 3310 cm⁻¹, which corresponds to the stretching frequency of —OH. Absorption in the region of 1614 cm⁻¹ corresponds to the C═O bond and carboxylate (COO) group of alginate. The infrared spectrum of alginate microspheres loaded with BSA showed the characteristic amide absorption band at 1660 cm⁻¹, which was due to the incorporated BSA in the microspheres. Chitosan-coated alginate microspheres displayed a characteristic absorption band for NH at ˜3300 cm⁻¹, which was masked by the broad peak of —OH. The infrared spectra of PCL also showed characteristic lactone band at 1740 cm⁻¹. The infrared absorbance profile of the PACE-A microspheres loaded with BSA displayed absorption bands for BSA (1550 cm⁻¹) alginate (1614 cm⁻¹), chitosan (3300 cm⁻¹) and PCL (1740 cm⁻¹) revealed the structure integrity of the microspheres and incorporated contents. Taken together, the infrared spectra of the microspheres shows that BSA was effectively incorporated in the microspheres having a primary coat of chitosan and secondary coat of PCL for the generation of PACE-A microspheres.

Thermal Analysis of Uncoated and Polymer-Coated Microspheres

Thermal analysis was carried out to confirm the cross-linking and its stability of the microspheres. An endothermic peak was observed in both coated or uncoated microspheres, which can be attributed to the dehydration of cross-linked polymers. The endothermic peak of alginate occurred at 109° C., due to dehydration as compared to the same microspheres loaded with BSA which gave an endothermic peak at 118° C. The stability of the chitosan-coated spheres improved as indicative of higher dehydration peak at 121.4° C. The same spheres coated with PCL had an even greater stability and dehydration peak that appeared at 127° C. At 270° C., a wide range of transition curves were present, which indicated the decomposition of alginate and chitosan biopolymers. In the case of PCL-coated microspheres, a sharp endothermic peak appeared at 360° C. due to the phase transition of PCL.

The thermo gravimetric analysis of sodium alginate, alginate microspheres, alginate microspheres loaded with BSA and coated with chitosan shows a first stage of 20% weight loss due to the elimination of water molecules. The complete decomposition of sodium alginate occurred at 250° C. with the elimination of carbon monoxide. In the case of alginate microspheres, the decomposition peak shifts from 250° C. to 290° C. The decomposition of chitosan coated PACE-A microspheres appeared around 290° C., but in the case of chitosan- and PCL-coated microspheres, the decomposition peak appeared at much higher temperature at 370° C., which indicates the increased stability of PACE-A microspheres.

Conformational Integrity of Entrapped BSA.

Circular dichroism analysis was performed to evaluate the conformational integrity of BSA during PACE-A microsphere formation. The standard or unencapsulated BSA and the BSA released from the PACE-A microspheres were virtually identical. Clearly circular dichroism analysis reveled that the helical peak and alpha helical structures of BSA remained intact when compared to control BSA and in contrast to currently available alginate microspheres of the prior art. This clearly indicates that the protein did not interact chemically with the matrix material. Moreover, these results also demonstrated that the method adopted for the encapsulation of BSA into microspheres did not lead to a significant irreversible aggregation or degradation of the carrier macromolecule (i.e., BSA).

Zeta Potential Measurements

Zeta potential is an important way to study the interactive properties of entrapped protein and the carrier system. The placebo microspheres show higher negative charges (−55.2±0.3 mV), in comparison with latex (−50.9±0.4) and poly lactide microsperes (−46.0). PACE-A microspheres loaded with BSA, displayed surface charges of −2.6±0.6 mV. This may be due to the net charges of the positively charged BSA with the negatively charged alginate matrix. Chitosan coating reduced the surface charge of microspheres to −6.6±0.3 mV. PCL also further reduced the surface charges, which clearly indicates that the PACE-A microspheres improved the stability of BSA.

In Vitro BSA Release From PACE-A Microspheres

In vitro release of BSA from uncoated alginate microspheres, chitosan-coated alginate microspheres (also with different amount of BSA) and PACE-A microspheres (coated with various concentrations of PCL) were carried out in PBS, pH 7.4 at 37° C. Even though single and double coating of chitosan improved the release pattern of BSA from alginate microspheres, chitosan triple coating resulted in a more efficiently controlled release of BSA. Subsequent studies were carried out with 4% chitosan dissolved in acetic acid coated alginate microspheres.

Effect of Protein Loading on Release Rates of BSA

The in vitro release profiles of chitosan-coated microspheres loaded with BSA (4.16%, 10.02% and 18.01%) by the direct method revealed a release rate of BSA (86% in 6 days) was more efficient in the case of 18.01% protein loaded in alginate microspheres. Microspheres loaded with 10.02% released 82.5% in 8 days and 4.04% BSA loaded microspheres released 81% in 9 days. These results indicated that the rate of release of BSA increased in proportion with protein loading percentage. Higher loading percentage of BSA led to burst release on day 1 in addition to an over-all higher rate of release.

The in vitro release data indicated that 79.4% of BSA was released in 16 days in the case of 5% PCL coated microspheres. On the other hand, 80.6% of BSA was released in 26 days in the case of 5% PCL double-coated PACE-A microspheres. Increasing the coating with a single coat of 10% PCL resulted in a prolonged release (33 days) of 78.6% of BSA from the microspheres. It can be noted that 5% single PCL coat PACE-A and 10% single PCL coat PACE-A released at approximately the same amount (78.6% and 80.2%) within a period of 16 and 33 days, respectively.

It can be interpreted from these results that a double coating of a concentrated PCL solution was more effective in retarding BSA release than a single coating of PCL of lesser concentration. It is also evident that 20% double coated PACE-A microspheres released 77.9% BSA in 90 days compared to 20% single coated PACE-A microspheres (76.2% in 55 days), as expected.

Microspheres of the invention may be particularly useful for administration of vaccines against diseases such as cholera, hepatitis, influenza, pneumonia and other diseases where the initial cite of infection and immune response are mucosal membranes. The microspheres may also be used for administration of therapeutic agents such as peptides, steroids, proteins and other agents wherein the preservation of conformational properties is desirable. For example, agents that target specific receptors are sometimes destroyed in the serum or other body fluids before they reach the receptors. The microspheres of the invention can be used as carriers for purpose of delivery to the target receptors.

The method of administration, whether systemic (parenteral administration, oral administration) or application to specific tissues (inhalation, instillation) will depend on the particular agent administered and the target tissue.

The microspheres of the invention may also be useful for application of agents such as pesticides and nutrients for agricultural purposes.

What we claim is:

1. A method of preparation microspheres comprising the steps of:
1) cross-linking alginate using epichlorohydrin;
2) forming microspheres from the product of step 1;
3) coating the product of step 2 with chitosan; and
4) coating the product of step 3 with PCL.

2. A microsphere comprised of alginate linked with epichlorohydrin, said microsphere being coated with chitosan.

3. The microsphere of claim 2 further being coated with PCL.

4. The microsphere of claim 3 having entrapped therein a vaccine.

5. The microsphere of claim 2 having entrapped therein a therapeutic agent.

6. The microsphere of claim 5 wherein the therapeutic agent is a protein or peptide.

7. The microsphere of claim 6 wherein the therapeutic agent is a peptide.

8. The microsphere of claim 3 wherein the therapeutic agent is a hormone.

9. A composition of matter comprising the microsphere of claim 3 in a pharmaceutically acceptable carrier.

10. The microsphere of claim 2 having entrapped therein a vaccine.
